(19) United States
(12) Patent Application Publication
(10) Pub. No.: US 2026/0207314 A1
(43) Pub. Date: Jul. 23, 2026

(54) HEAD FIXATION DEVICE

(71) Applicant: The Cleveland Clinic Foundation, Cleveland, OH (US)

(72) Inventors: Jakov Tiefenbach, Cleveland, OH (US); Kenneth B. Baker, Chesterland, OH (US); Mark S. Lobosky, Chardon, OH (US); Nicole Byram, Cleveland, OH (US)

(21) Appl. No.: 19/157,779

(22) PCT Filed: Apr. 25, 2024
(86) PCT No.: PCT/US2024/026196

§ 371 Date: Aug. 19, 2025

Related U.S. Application Data
(60) Provisional application No. 63/461,932, filed on Apr. 26, 2023.

(51) Int. Cl. A61D 3/00

(52) U.S. Cl. CPC A61D 3/00; A61D 2003/003

(57) ABSTRACT

A head fixation device for an animal subject includes a base for supporting the animal subject and a head fixture fixed to the base, the head fixture defining a cavity for receiving a snout of the animal subject. The head fixation device further includes first and second opposing set screws configured to compressively engage a neck of the animal subject when received in the head fixation device in order to restrain neck movement thereof.

### CROSS-REFERENCE TO RELATED APPLICATIONS

This application claims the benefit of U.S. provisional application Ser. No. 63/461,932 filed Apr. 26, 2023, the contents of which are incorporated by reference.

### STATEMENT OF GOVERNMENT-SPONSORED RESEARCH

This invention was made with government support under NS116384 awarded by the National Institutes of Health. The government has certain rights in the invention.

### TECHNICAL FIELD

The present disclosure relates to a head fixation device and more particularly, to a head fixation device for restraining the head of an awake animal subject during data collection.

### BACKGROUND

The value of neuroimaging methods in neuroscience research has grown significantly in recent decades. Techniques such as functional magnetic resonance imaging (fMRI), magnetic resonance spectroscopy (MRS), diffusion tensor imaging (DTI), and positron emission tomography (PET) provide a non-invasive method of exploring the structure and function of nervous tissue. These advanced imaging techniques have played a crucial role in advancing many fundamental areas of neuroscience, including the study of multiple sclerosis, Parkinson's disease, Alzheimer's disease, traumatic brain injury, and other neurological conditions.

A major barrier to applying some of these advanced techniques in preclinical animal models is the necessity to minimize head motion during image acquisition. Animal data can be acquired under general anesthesia to ensure the animal's head remains still throughout the imaging sequence. But anesthesia can have profound effects on brain metabolism and selectively alter dynamic neural networks by reducing functional connectivity within the frontotemporal association cortex and higher-order thalamocortical networks. Additionally, anesthetics can introduce confounding factors in fMRI by interfering with neuronal activity, cerebral vasculature, and neurovascular coupling. Similarly, the use of anesthetics can affect cerebral metabolic rate and brain metabolite concentration, directly influencing the results of MR spectroscopy. Therefore, to optimize data quality and ensure an accurate representation of neurological processes within a living animal, it is ideal to acquire functional brain images in fully awake and alert animals.

Over the years, numerous research groups have made efforts to obtain brain images in awake rodents, albeit with varying degrees of success. Some groups have explored the implantation of a head fixation clamp within the animal's skull, which can be secured to a fixed plate. While this approach yielded promising results, it carries the risk of head injury and proves highly impractical for experiments requiring further manipulation of the skull. Other groups have evaluated the use of muscle relaxants to prevent movement during image acquisition. However, this method necessitates animal intubation and ventilation, which have been associated with significant morbidity and mortality.

### BRIEF SUMMARY OF THE INVENTION

According to a first aspect, a head fixation device for an animal subject includes a base for supporting the animal subject and a head fixture fixed to the base, the head fixture defining a cavity for receiving a snout of the animal subject. The head fixation device further includes first and second opposing set screws configured to compressively engage a neck of the animal subject when received in the head fixation device in order to restrain neck movement thereof.

According to a second aspect, a head fixation device for an animal subject includes a base for supporting the animal subject; a head fixture fixed to the base, the head fixture defining a cavity for receiving a snout of the animal subject; first and second opposing set screws configured to compressively engage a neck of the animal subject when received in the head fixation device in order to restrain neck movement thereof; and an adjustable toothbar configured to follow a planetary path about a pivot axis in order to adjust a position of the animal's head within the head fixture in-use. The base defines a plurality of strap apertures configured to receive a flexible strap for restraining a body of the animal subject.

### BRIEF DESCRIPTION OF THE DRAWINGS

FIG. 1 is a perspective view of an example head fixation device;

FIG. 2 is an enlarged perspective view of the head fixation device focusing on a head-fixation portion thereof; and

FIG. 3 is a rear perspective view of the head fixation device in which a carriage of the head fixation device has been removed.

### DETAILED DESCRIPTION

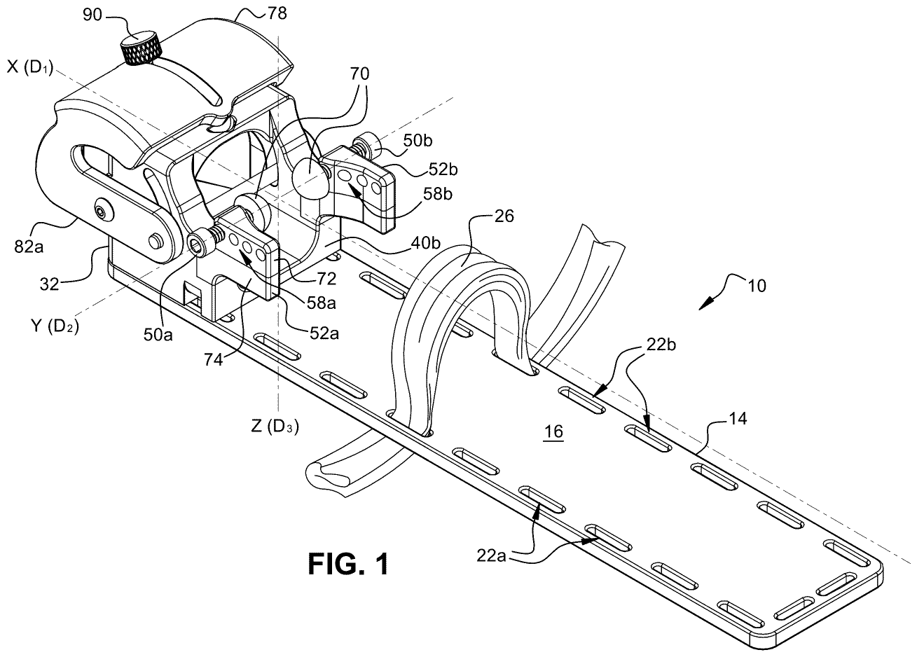

Turning to FIG. 1, an example head fixation device 10 for securing an animal subject's head position will now be described. The device 10 is particularly useful for restraining rats (e.g., between 200 and 450 grams in weight) during data collection with an MRI machine. However, the device 10 may be used with other animal subjects and/or for other types of data collection without departing from the scope of the disclosure.

The device 10 is arranged relative to first, second, and third imaginary axes X, Y, Z that are perpendicular to each other and respectively define longitudinal, lateral, and vertical directions D₁, D₂, D₃ relative to the device 10. The device 10 comprises a rigid base 14 defining a planar support surface 16 for supporting the animal subject. The support surface 16 is elongated in the longitudinal direction D₁ and substantially parallel to the longitudinal and lateral directions D₁, D₂. Moreover, the base 14 defines a plurality of strap apertures 22a, 22b that extend through the support surface 16 and allow for attachment of a flexible strap 26 to restrain a body of the animal subject. In the present example, the flexible strap 26 comprises a strip of gauze, although other flexible materials may be used in other examples.

The plurality of strap apertures 22a, 22b includes a first set of apertures 22a and a second set of apertures 22b, wherein the apertures 22a, 22b in each set are aligned along (i.e., substantially parallel to) the longitudinal direction D₁, adjacent to respective lateral edges of the support surface 16. Additionally, each aperture 22a in the first set is aligned with a corresponding aperture 22b in the second set along the lateral direction D₂. Accordingly, the flexible strap 26 can be fed through a pair of corresponding apertures 22a, 22b to restrain a body of the animal subject against the support surface 16 between the strap 26 and the base 14.

Moreover, the location in which the strap 26 engages and restrains the animal subject along the longitudinal direction DI can be adjusted by feeding the strap 26 through different pairs of corresponding apertures 22a, 22b.

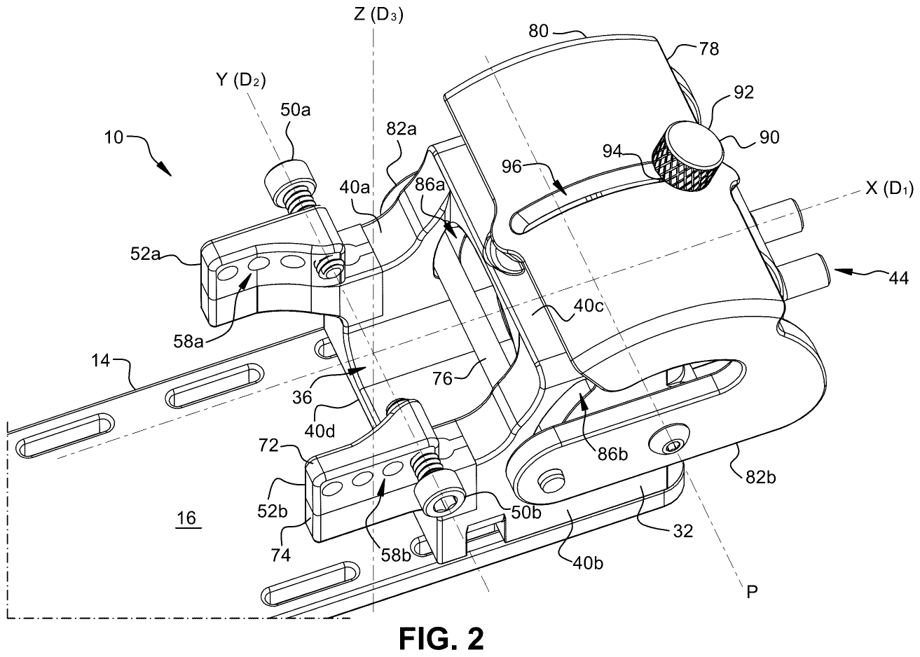

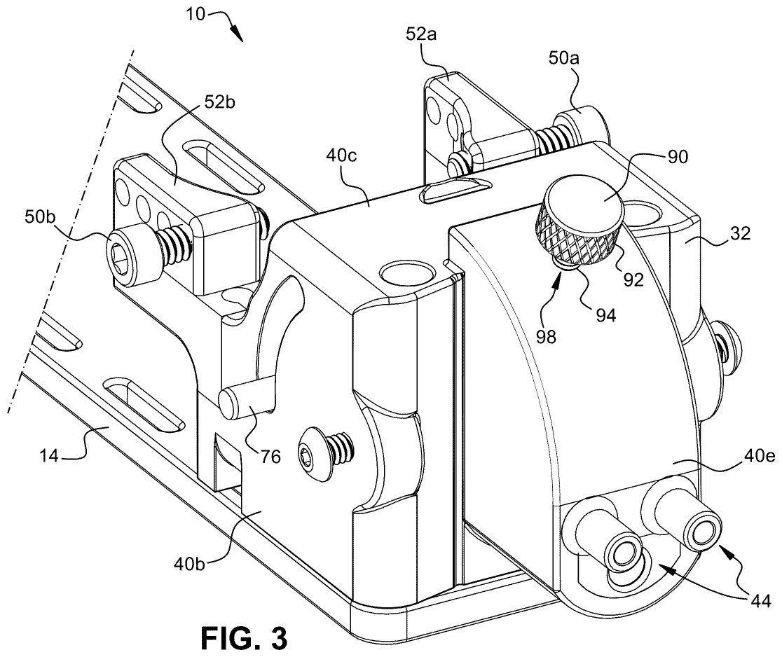

As shown best in FIG. 2, the device 10 further includes a rigid head fixture 32 that is fixed to the base 14 and defines a cavity 36 for receiving a snout of the animal subject. In particular, the fixture 32 comprises first and second sidewalls 40a, 40b that can limit movement of the snout along the lateral direction D₂, and upper and lower walls 40c, 40d that can limit movement of the snout along the vertical direction D₃. Moreover, the fixture 32 comprises an anterior wall 40e (see FIG. 3) that can limit anterior movement of the snout along the longitudinal direction D₁. The anterior wall 40e of the fixture 32 also defines a plurality of fluid ports 44, which may be used for delivering anesthesia or other fluids to/from the animal subject.

The device 10 further includes first and second set screws 50a, 50b that are adjustably coupled to the fixture 32 as shown (or optionally directly to the base 14 or to other structure fixedly attached to the base-not shown) for restraining movement of the subject's neck along the lateral direction D₂. More specifically, the device 10 includes first and second arms 52a, 52b that are respectively fixed to and extend posteriorly from the first and second sidewalls 40a, 40b of the fixture 32 along the longitudinal direction D₁. The first arm 52a defines a first set of threaded apertures 58a for threadably receiving the first set screw 50a, and the second arm 52b defines a second set of threaded apertures 58b for threadably receiving the second set screw 50b. The threaded apertures 58a, 58b in each set are aligned along the longitudinal direction D₁, such that their central axes are substantially parallel to each other along the lateral direction D₂. Additionally, each aperture 58a in the first set is coaxially aligned with a corresponding aperture 58b in the second set.

The respective first and second set screws 50a, 50b can be threadably received through a coaxial pair of corresponding apertures 58a, 58b to define a space therebetween for accommodating and immobilizing the neck of the animal subject via compressive engagement between the opposing set screws 50a, 50b. In this manner, movement of the animal's neck along the lateral direction D₂ is inhibited. Moreover, the location in which the set screws 50a, 50b engage the neck along the longitudinal direction D₁ can be adjusted by selecting an appropriate pair of coaxial apertures 58a, 58b along the arms 52a, 52b. Optionally, each set screw 50a, 50b can include a pad 70 of soft material (e.g., polyvinyl chloride, polyurethane, etc.) attached to and covering a distal tip of its threaded shank to minimize discomfort to the animal subject where compressively engaged against its neck.

Each arm 52a, 52b comprises a first portion 70 that defines its associated screw apertures 58a, 58b, and a second portion 72 that couples the first portion 70 to the fixture 32. The first portion 70 is relatively harder than the second portion 72 in order to provide rigid threads for engagement with the set screws 50a, 50b. Meanwhile, the second portion 72 is relatively more elastic than the first portion 70 and fixture 32 such that the arms 52a, 52b can flex to help conform to the animal subject's neck. The relative hardness and elasticity of the first and second portions 70, 72 can be achieved in a variety of manners. For example, in the present embodiment, the fixture 32 and arms 52 a, 52 b are integrally formed via a 3D printing process that adjusts the composition of the first and second portions 70, 72 and fixture 32 such that they have different hardness and elasticity. In other examples, the first and second portions 70, 72 and fixture 32 can be separately formed with different materials having different hardness and elasticity.

The device 10 includes a rigid toothbar 76 in the form of a bar (optionally cylindrical) that can be located within the cavity 36 of the fixture 32 and extends laterally. The toothbar 76 is configured so that at least the animal's upper jaw can be positioned over it so that its upper teeth are confined anteriorly against the toothbar 76 when the animal is engaged within the device 10, to help restrain its head. Specifically, the snout of the animal subject can be placed within the cavity 36 such that the upper and lower jaws of the subject close about the toothbar 76. As discussed further below, the toothbar 76 is adjustably coupled to the base 14 such that its position along the longitudinal and/or vertical directions D₁, D₃ can be adjusted to adjust the orientation of the animal's head in order to optimize it for imaging.

More specifically, the toothbar 76 is fixed to a rigid carriage 78, which is pivotally coupled to the fixture 32. The carriage 78 includes a canopy 80 disposed above the fixture 32, and first and second pivot arms 82a, 82b parallel to one another and extending from opposite sides of the canopy 78. The pivot arms 82a, 82b are pivotally coupled to the first and second sidewalls 40a, 40b of the fixture 32, respectively, and configured to pivot about a pivot axis P that extends laterally and is substantially parallel to the toothbar 76. Moreover, the toothbar 76 extends through first and second opposing planetary channels 86a, 86b defined respectively in the first and second sidewalls 40a, 40b of the fixture 32, such that the ends of the toothbar 76 are fixed to the first and second pivot arms 82a, 82b of the carriage 78. As the carriage 78 pivots about the pivot axis P, the toothbar 76 affixed between ends of the opposing pivot arms 82a, 82b is caused to follow a planetary path through the channels 86a, 86b, about and coaxial with the pivot axis P, whose radius corresponds to the distance between the pivot axis P and the toothbar 76.

The device 10 further comprises a fixation screw 90 for selectively fixing the carriage 78 and toothbar 76 relative to the fixture 32 (and to the base 14 attached thereto). In particular, the fixation screw 90 comprises a head 92 and a threaded shank 94 that extends through a guide channel 96 of the carriage 78 and engages a threaded aperture 98 (see FIG. 3) defined by the fixture 32. The fixation screw 90 can be threadably advanced into/through the threaded aperture 98 until its head 92 engages the carriage 78, thereby inhibiting movement of the carriage 78 and toothbar 76 relative to the fixture 32.

Moreover, when its desirable to adjust a position of the toothbar 76, the fixation screw 90 can be loosened to permit movement of the carriage 78 and toothbar 76 relative to the fixture 32, and then re-tightened once a desirable position has been achieved. Preferably, the canopy 80 and guide channel 96 of the carriage 78 are curved such that their axes of curvatures substantially coincide with the pivot axis P, thereby enabling the carriage 78 to pivot about the pivot axis P.

The first and second set screws 50a, 50b and the toothbar 76 as described above are adjustable to accommodate different animal subjects and/or fixation configurations. As shown, the first and second set screws 50a, 50b are adjustably coupled to the fixture 32 via threaded engagement with arms 52a, 52b that extend therefrom, and the toothbar 76 is adjustably coupled to the fixture 32 via a pivotable carriage 78. However, the first and second screws 50a, 50b and/or toothbar 76 may be adjustably coupled directly to the base 14 (not shown) in an alternative embodiment. For instance, the first and second set screws 50a, 50b can be threadably coupled directly to the base 14 (or to arms that extend directly therefrom). As another example, the carriage 78 for the toothbar 76 can be directly coupled to the base 14 instead of to the fixture 32.

The device 10 as described above has various features for restricting movement of the subject animal's head. Moreover, the components of the device 10 (e.g., base 14, fixture 32, set screws 50a, 50b, toothbar 76, carriage 78) preferably are made of a non-metallic material such as plastic (e.g., polyethylene, polyoxymethylene, polypropylene, polystyrene, polyvinyl chloride, etc.) that is suitable for use with an MRI machine. Polyoxymethylene is particularly useful for the toothbar 76 for its high wear-resistance, strength, and stiffness. Preferably, the device 10 does not include any metal.

### EXAMPLE

A device 10 as described was used in an experiment involving eight naïve male rats (Long-Evans, 225 +/−25 g). The rats were anesthetized using 2% isoflurane and carefully placed into the device 10. Then 150 μL of venous blood was drawn from the tail vein to obtain a baseline, pre-scan corticosterone measurement. Subsequently, the device 10 was inserted into a 7T BRUKER BioSpec 70/20 USR MRI System. To monitor the rat's vital signs, a respiratory probe was securely taped to its body and a fiber optic pulse oxygen meter was attached to the hind paw. Finally, a thirty-millimeter surface coil was placed over the rat's scalp to enhance image quality. Once the subject was stable and exhibited appropriate physiological parameters, an MRI localizer scan was acquired, and the position within the scanner readjusted as necessary. This was followed by BO map to quantify and correct subject-induced geometric distortions. Lastly, high-resolution anatomic data were acquired using a 3D TurboRARE with 60 0.2 mm thick slices (TE=36 ms; TR=1400 ms; RARE factor=8; FOV=30×35 mm2; matrix=150×175; in-plane resolution=0.2 ×0.2 mm2).

Upon completion of the of the aforementioned steps (requiring between seven to ten minutes), the delivery of isoflurane was stopped, and a five-minute count began. At the conclusion of the five-minute countdown, three consecutive whole-brain fMRI scans were acquired, each scan lasting 10 min and 24 s. The scans were acquired using segmented spin-echo, echo planar imaging with 35 contiguous 0.8 mm thick axial slices (echo time (TE)=15 ms; repetition time (TR)=1040 ms; flip angle (FA)=90°; field of view (FOV)=35×18 mm2; matrix=125×64; in-plane resolution=0.28×0.28 mm2; 200 vol). Following a total period of 31 min and 12 sec of awake fMRI scan acquisition, isoflurane (1.5 %) administration was resumed and 150 μL of venous blood was again drawn from the tail vein to obtain a post-scan corticosterone measurement. Thereafter, anesthesia was discontinued and the animal was carefully removed from the device 10.

The fMRI scans obtained in the experiment were used to quantify the level of motion during the awake stage. Across the three fMRI scans, a total of 600 volumes were acquired, each providing a ~3.12 second snapshot of the rat's spatial position in relation to the center of the MRI scanner. The volumes that were significantly affected by the motion artifact on visual inspection, or displayed a relative displacement of >0.28 mm (i.e., the size of the smallest voxel dimension), were identified and replaced with a “dummy volume” (i.e., an exact copy of the preceding volume) as per established research protocols. The MCFLIRT algorithm was employed to evaluate the extent of motion between each acquired volume. The analysis focused on determining the mean absolute and relative displacement, with the former reflecting the overall displacement of the subject during the entire imaging session, and the latter representing the average displacement between individual fMRI volumes. Additionally, the extent of rotation and translation across all three planes were examined throughout the scan acquisition.

The number of fMRI volumes that had to be replaced with “dummy volumes” due to motion artifact can serve as index of the overall motion contamination present throughout the study. The number of motion-contaminated volumes ranged from 4 to 11 across the eight animals tested, representing 0.6 % to 1.8 % of the total volumes acquired.

After the exclusion of the motion affected volumes, the MCFLIRT algorithm was utilized to assess the overall level of motion between each individual volume across the entire awake acquisition. The mean absolute and relative displacement across the entire cohort were 0.0256 (SD: 0.011) and 0.009 (SD: 0.002) millimeters, respectively. There was no statistically significant difference in the total displacement across the three fMRI scanning sessions (ANOVA, p=0.27).

Moreover, serum corticosterone levels were assessed both before and after the awake scan acquisition to gauge the extent of stress induced by the scanning process. Among the subjects, the average baseline serum corticosterone level stood at 210.74 ng/ml, increasing slightly to 216.52 ng/ml by the conclusion of the scan. Notably, there was no statistically significant disparity in cortisol values between the scan's onset and conclusion, as indicated by a paired t-test (p=0.159).

Finally, none of the rats suffered mortality or displayed any signs of disability throughout the course of the experiment.

In summary, the experiment assessed the utility of the head fixation device 10 for conducting awake scans in rats. The device 10 demonstrated effective motion control, facilitating the acquisition of high-quality awake brain scans while minimizing the impact on animals'stress levels.

The invention has been described with reference to the example embodiments described above. Modifications and alterations will occur to others upon a reading and understanding of this specification. Example embodiments incorporating one or more aspects of the invention are intended to include all such modifications and alterations insofar as they come within the scope of the appended claims.

1. A head fixation device for an animal subject, comprising:
a base for supporting the animal subject;
a head fixture fixed to the base, the head fixture defining a cavity for receiving a snout of the animal subject;
a first arm and a second arm extending from opposing sides of the head fixture, wherein each arm comprises a first portion and a second portion that couples the first portion to the head fixture; and
first and second opposing set screws configured to engage with the first portions of the first and second arms and configured to compressively engage a neck of the animal subject when received in the head fixation device in order to restrain neck movement thereof,
wherein the first portion of the first arm is harder and less elastic than the second portion of the first arm.

2. The head fixation device of claim 1, further comprising a first set of threaded apertures in the first arm for threadably receiving the first set screw; and a second set of threaded apertures in the second arm for threadably receiving the second set screw, wherein opposing pairs of apertures within the first and second sets thereof are coaxially aligned.

3. The head fixation device of claim 2, wherein the first portion of the first arm defines the first set of threaded apertures, and wherein the first portion of the second arm defines the second set of threaded apertures.

4. The head fixation device of claim 1, wherein:
the first portion of the second arm is harder and less elastic than the second portion of the second arm.

5. The head fixation device of claim 1, further comprising a toothbar configured to be engaged by a jaw of the animal subject.

6. The head fixation device of claim 5, wherein the toothbar is configured to follow a planetary path about a pivot axis in order to adjust a position of the animal's head in-use.

7. The head fixation device of claim 6, further comprising a carriage that is pivotally coupled to the housing such that the carriage can pivot about the pivot axis, wherein the toothbar is fixed to the carriage and caused to correspondingly follow said planetary path upon pivoting of the carriage.

8. The head fixation device of claim 7, wherein:
the head fixture comprises a first sidewall that defines a first planetary channel and a second sidewall that defines a second planetary channel,
the carriage comprises a first pivot arm that is pivotally coupled to the first sidewall and a second pivot arm that is pivotally coupled to the second sidewall, and
the toothbar extends through the first and second planetary channels and is fixed to the first and second pivot arms.

9. The head fixation device of claim 7, further comprising a fixation screw for selectively fixing the carriage relative to the head fixture.

10. The head fixation device of claim 9, wherein:
the head fixture defines a threaded aperture,
the carriage defines a guide channel, and
the fixation screw extends through the guide channel and threadably engages within the threaded aperture.

11. The head fixation device of claim 10, wherein the guide channel is curved such that an axis of curvature for the guide channel substantially coincides with the pivot axis.

12. The head fixation device of claim 1, wherein the base defines a first strap aperture and a second strap aperture that are configured to receive a flexible strap for restraining a body of the animal subject.

13. The head fixation device of claim 1, wherein:
the base defines a first set of strap apertures and a second set of strap apertures, the strap apertures in each set being aligned along a longitudinal direction, and
each strap aperture of the first set of strap apertures is aligned with a corresponding strap aperture of the second set of strap apertures.

14. The head fixation device of claim 1, wherein the first set screw and the second set screw each comprise a soft pad attached to a distal tip of a shank thereof and configured to minimize discomfort when compressively engaged against the animal's neck in-use.

15. The head fixation device of claim 1, wherein the base, the head fixture, the first set screw, and the second set screw are not made of metal.

16. The head fixation device of claim 1, wherein the head fixation device contains no metal.

17. A head fixation device for an animal subject, comprising:
a base for supporting the animal subject;
a head fixture fixed to the base, the head fixture defining a cavity for receiving a snout of the animal subject;
first and second opposing set screws configured to compressively engage a neck of the animal subject when received in the head fixation device in order to restrain neck movement thereof; and
an adjustable toothbar configured to follow a planetary path about a pivot axis in order to adjust a position of the animal's head within the head fixture in-use.

18. The head fixation device of claim 17, further comprising a first set of threaded apertures for threadably receiving the first set screw and a second set of threaded apertures for threadably receiving the second set screw, wherein opposing pairs of apertures within the first and second sets thereof are coaxially aligned.

19. The head fixation device of claim 17, wherein the base defines a plurality of strap apertures configured to receive a flexible strap for restraining a body of the animal subject.

20. The head fixation device of claim 17, further comprising a first arm and a second arm extending from opposing sides of the head fixture, wherein each arm comprises a first portion and a second portion that couples the first portion to the head fixture, wherein the first and second opposing set screws are configured to engage with the first portions of the respective first and second arms, wherein the first portion of the first arm is harder and less elastic than the second portion of the first arm.

21. The head fixation device of claim 17, further comprising a carriage that is pivotally coupled to the housing such that the carriage can pivot about the pivot axis, wherein the toothbar is fixed to the carriage and caused to correspondingly follow said planetary path upon pivoting of the carriage.
